(19) United States
(12) Patent Application Publication
(10) Pub. No.: US 2023/0338114 A1
(43) Pub. Date: Oct. 26, 2023

(54) SHAPE OPTIMIZED PREPARATION AND RESTORATION DEVICES, SYSTEMS, AND METHODS FOR DENTAL AND OTHER APPLICATIONS

(71) Applicants: REGENTS OF THE UNIVERSITY OF MINNESOTA, Minneapolis, MN (US); REGENTS OF THE UNIVERSITY OF MINNESOTA, Minneapolis, MN (US)

(72) Inventors: Alex Siu Lun FOK, Plymouth, MN (US); Yung-Chung CHEN, II, Tainan City (TW); Jose Antonio OLIVARES TREVINO, San Nicolas de los Garza (MX); Hooi Pin CHEW, Bloomington, MN (US)

(21) Appl. No.: 18/027,576

(22) PCT Filed: Sep. 22, 2021
(86) PCT No.: PCT/US2021/071554

§ 371 Date: Mar. 21, 2023

Related U.S. Application Data
(60) Provisional application No. 62/706,976, filed on Sep. 22, 2020.

(51) Int. Cl. A61C 1/08; A61C 13/34; A61C 9/00; G06F 30/23; G16H 50/50

(52) U.S. Cl. CPC A61C 1/082; A61C 13/34; A61C 9/0053; A61C 9/0006; G06F 30/23; G16H 50/50

(57) ABSTRACT

A dental preparation block guide comprises a chamber that is customized to receive at least one tooth of a patient for a shape-optimized restoration of the at least one tooth; and a channel formed in a surface of the block guide at a location selected relative to the shape-optimized restoration to be performed, the channel comprising an aperture extending through the surface to the chamber such that a dental handpiece can be inserted into and guided within the channel while a tool of the dental handpiece can interact with the at least one tooth in the chamber through the aperture to form a shape-optimized preparation in the at least one tooth to receive the shape-optimized restoration. Related methods and system, as well as other devices, also are disclosed.

### RELATED APPLICATION

The present application claims the benefit of U.S. Provisional Application No. 62/706,976 filed Sep. 22, 2020, which is hereby incorporated herein in its entirety by reference.

### TECHNICAL FIELD

The present disclosure relates generally to dental applications, and more particularly to preparation guides used to facilitate dental restorations.

### BACKGROUND

Millions of dental restorations are placed in patients every day. These dental restorations may take the form of direct restorations, commonly referred to as fillings, which involve preparing the tooth by removing the carious or damaged tooth tissues and forming a restoration in the prepared tooth intra-orally. Alternatively, dental restorations may be indirect, which requires fabricating part of the restoration outside of the mouth, such as is often the case with veneers, crowns, inlays, caps, or bridges. In both cases, the tooth or teeth that support the restoration will need to be prepared. The supporting portion of the tooth or teeth (e.g., after the tooth is drilled or cut to remove the damaged or decayed portion) is referred to as a preparation.

Historically, dentists have relied on their own judgement when preparing the tooth for dental restoration. This requires the dentist to examine the tooth and visually determine what sections of the tooth to remove before restoring. It is difficult for dentists to visually analyze the tooth and prepare it by hand in a way that will minimize stress on the restoration, supporting tooth or teeth, and their interfaces from chewing. Because this practice relies on the individual dentist, it takes time for preparation and there is room for error. Prolonged preparation can incur trauma to the pulp tissues of the tooth. Indirect restorations are also designed and fabricated days or even weeks after the preparation is made. Many dental restorations later fail through fracture, debonding, interfacial leakage, or recurrent caries from excessive masticatory stresses.

In order to develop a more precise practice, some dentists have used a preparation guide to prepare the tooth for indirect restoration. These guides include a block that rests around the tooth and provides the dentist with a channel that guides cutting. Dental preparation guides are roadmaps that direct dentists in preparing the tooth for restoration. However, conventional preparations are box-shaped and are not shape-optimized. Because of this, box-shaped preparations often lead to excessive stress on the restoration, supporting teeth, and their interfaces, which results in failure of the restoration.

Shape-optimized preparation has a more organic geometry which minimizes interfacial and bulk stresses. This in turn reduces the likelihood of restoration failure. Because of its organic geometry, however, shape-optimized preparations are difficult, if not impossible, to prepare by hand accurately. Additionally, preparations for direct restorations are still made by hand without a guide. Thus, there is a need for a dental restoration preparation guide for shape-optimized preparations for both direct and indirect restorations.

Moreover, having a preparation guide is very pertinent in the current COVID-19 pandemic environment. Reopening elective dental procedures bears the risk of exposing dental professionals and patients to SARS-COV-2. In order to ensure no or little nosocomial transmission of SARS-COV-2 (and other aerosol- or air-borne viruses) in the dental operatory setting, there is a demand for procedures that generate less aerosol and require fewer clinic visits.

### SUMMARY

Therefore, there is a need for a dental preparation and restoration system that provides shape-optimization for both preparation and restoration, to reduce failure of dental restorations by minimizing interfacial and bulk stresses. There also is a need for a dental restoration preparation and restoration system that reduces visits and minimizes the generation of aerosol to protect all individuals involved in the dental restoration.

In one embodiment, a dental preparation block guide comprises a chamber that is customized to receive at least one tooth of a patient for a shape-optimized restoration of the at least one tooth; and a channel formed in a surface of the block guide at a location selected relative to the shape-optimized restoration to be performed, the channel comprising an aperture extending through the surface to the chamber such that a dental handpiece can be inserted into and guided within the channel while a tool of the dental handpiece can interact with the at least one tooth in the chamber through the aperture to form a shape-optimized preparation in the at least one tooth to receive the shape-optimized restoration.

In another embodiment, a method of providing a dental restoration in at least one tooth of a patient comprises applying a finite element analysis operating on a computer system to a model of the at least one tooth of the patient to design a shape-optimized preparation in the at least one tooth of the patient; and applying the finite element analysis operating on the computer system to the shape-optimized preparation in the at least one tooth of the patient to create a shape-optimized restoration to be applied to the shape-optimized preparation in the at least one tooth of the patient.

Embodiments of the disclosure provide dentists and other practitioners with the ability to conduct a shape optimized direct or indirect restoration. For example, one embodiment may be used in the preparation of cavity restorations, although this and other embodiments could also be employed during other direct or indirect dental restoration procedures.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of an embodiment of a shape-optimized restoration preparation system, located in a mouth of a patient for dental restoration.

FIG. 2 is a perspective view of a dental handpiece according to an embodiment of the disclosure.

FIG. 3 is a perspective view of an embodiment of a block guide of the disclosure.

FIG. 4 is a flowchart of an embodiment of a method of the disclosure.

FIGS. 5A-F depict construction of a physical model for a mandibular first molar attached with a second-premolar pontic according to one example study.

FIG. 6 is a finite element model of a 2-unit cantilevered bridge according to one example study.

FIG. 7 is a conventional design of a 2-unit cantilevered bridge with box-shaped preparation and fiber reinforcement in the restoration.

FIGS. 8A-B depict results from a first step of a stress-induced material transformation (SMT) optimization of a cavity/retainer design according to one example study.

FIG. 9A is a graph of optimized cavity volume versus assumed failure stress according to one example study.

FIG. 9B is a graph of optimized fiber volume in restoration versus assumed failure stress according to one example study.

FIGS. 10A-C depict results from a second step of SMT optimization of the fiber layout according to one example study.

FIGS. 11A-C depicts maximum principal stress profiles of conventional and optimized designs without embedded fibers according to one example study.

FIGS. 12A-B depicts maximum principal stress profiles of conventional and optimized designs with embedded fibers according to one example study.

FIG. 13A depicts normal stress distribution at a tooth-restoration interface with conventional (left) and optimized (right) designs according to one example study.

FIG. 13B depicts maximum principal stress distributions within the tooth of FIG. 13A with conventional (left) and optimized (right) designs.

FIGS. 14A-C depict a fiber layout in a conventional 2-unit cantilevered FRC bridge. While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Embodiments are directed to shape-optimized dental restoration preparation guides. A purpose of shape-optimized dental preparation guides is to allow dentists to easily create specific restorations that are less likely to fail than conventional restorations created without shape-optimized dental preparation guides. By using shape-optimization technology to develop the preparation guide, the dental restoration can limit interfacial and bulk stresses. If not addressed, these stresses may lead to shortened restoration life or complete failure. By providing a preparation guide for shape-optimized preparations and restorations, the restorations can be more efficient, precise, and durable.

The use of shape-optimized dental preparation guides according to the disclosure also can have the benefit of reducing the number of patient visits necessary to complete a dental restoration, while also reducing damage to the prepared tooth and practitioner exposure to aerosol generation via shorter patient visits. In embodiments, the shape-optimization is algorithmically created specific to the patient and the restoration type.

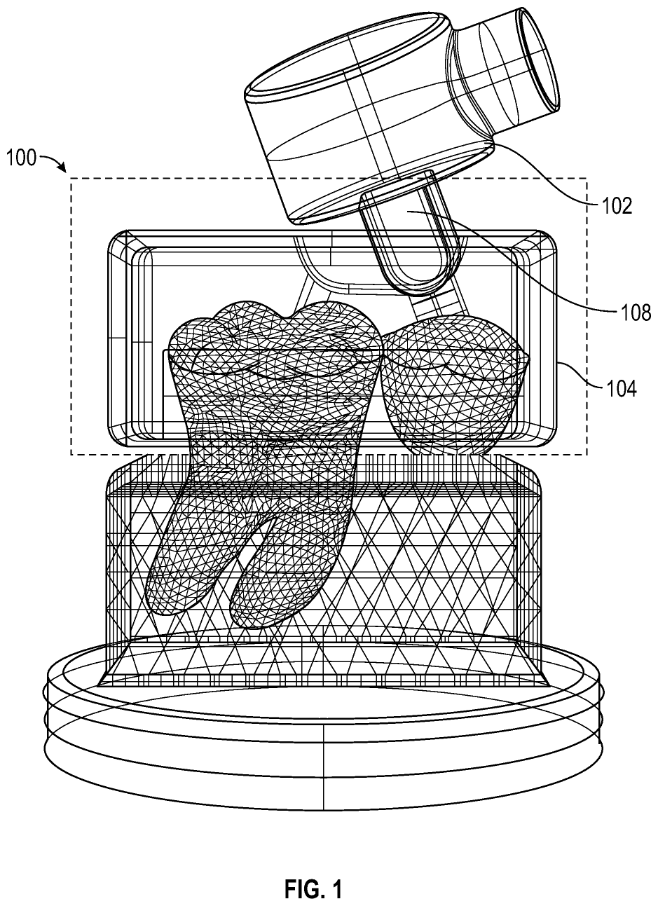

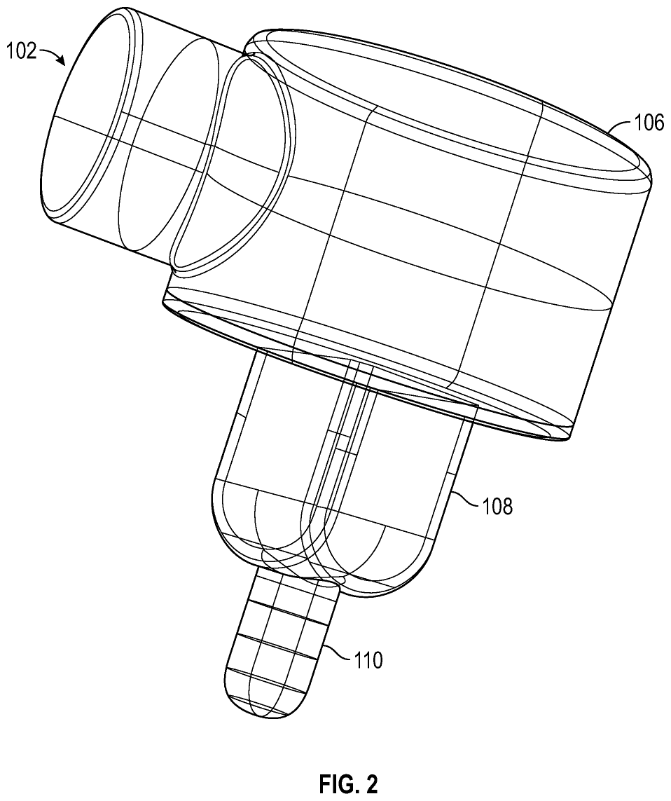

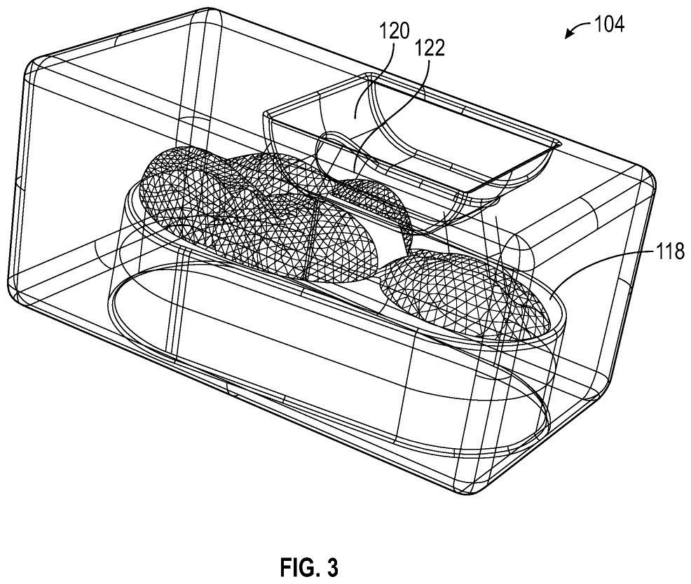

FIG. 1 depicts an embodiment of a preparation guide system 100 and a dental handpiece 102 (further depicted in FIG. 2). Preparation guide system 100 comprises a block guide 104 (further depicted in FIG. 3) and an adaptor 108 (further depicted in FIG. 2). Block guide 104 and adaptor 108 are complementary and interact with one another to facilitate creation of a particular dental preparation for receiving a particular dental restoration.

Referring to FIG. 2, dental handpiece 102 comprises a body 106, adaptor 108, and a tool 110 (such as a rotary cutting instrument depicted in FIG. 2). Dental handpiece 102 can comprise other components and features as known to be part of conventional dental handpieces, such as a handle (not shown), in various embodiments. For example, dental handpiece 102 can comprise one or more of a variety of different devices in various embodiments, such as an imaging device or camera (such as a wired or wireless camera, including a camera that can enable live viewing of an image), light, drill, scraper, cutting tool, pick, water spout, air nozzle, contact stylus, or some other dental or medical device. In general, however, dental handpiece 102 can include or be fitted with one or more of any devices that interact with the tooth for the dental restoration preparation and are configured to do so via adaptor 108 and block guide 104, as discussed in more detail below.

Adaptor 108 is designed to interact with block guide 104 such that dental handpiece 102 can be manipulated by a dentist or other practitioner to bring tool 110 in proximity to the patient's tooth or teeth requiring preparation(s) and/or restoration(s), guided by block guide 104. In some embodiments, adaptor 108 has a standardized shape and configuration, while in other embodiments adaptor 108 itself is also adapted or formed to interact with block guide 104. For example, adaptor 108 can be a custom component created for a particular patient and restoration and configured to fit onto dental handpiece 110. Adaptor 108 additionally can be customized to fit a particular dental handpiece 102. In some embodiments, other components of preparation guide system 100, or devices that interact with preparation guide system 100, also can be custom components configured to fit onto or otherwise interact with one or more dental handpieces.

Referring also to FIG. 3, block guide 104 can comprise a block-like structure as depicted, having a chamber 118 configured to fit on or over one or a plurality of teeth. In another embodiment, block guide 104 can comprise a substantially U-shaped structure configured to fit on or over one or a plurality of teeth without also fitting between any of the teeth. In yet another embodiment, block guide 104 can comprise a retainer-like device configured to fit over all of the upper or lower teeth of a patient. In a further embodiment, block guide 104 can comprise a bridge-like structure configured to fit over one or a plurality of teeth on each side of a patient's mouth, on the top or bottom. In a still further embodiment, block guide 104 can fit between at least one upper tooth and at least one lower tooth, such as to enable a dentist to view a bite surface between teeth requiring restoration. Still other configurations of block guide 104 also are possible, as block guide 104 can be customized according to a particular restoration or medical need.

In particular, block guide 104 comprises patient-customized chamber 118 for the tooth or teeth of a patient. Thus, chamber 118 is formed for a particular patient's tooth or teeth (such as from a dental impression) and can be customized for the particular restoration to be performed on the patient's tooth or teeth.

Block guide 104 also comprises a customized channel 120 that is configured to guide dental handpiece 102 (via adaptor 108) into and within block guide 104 such that tool 110 can be brought proximate the tooth or teeth via tool aperture 122 formed within channel 120. The complementary forms of adaptor 108 and channel 120 enable a dentist or other practitioners to manipulate dental handpiece 102 as adaptor 108 is guided within channel 120, thereby manipulating tool 110 (such as a rotary cutting instrument) proximate the tooth or teeth in chamber 118 to make the shape-optimized cavity preparation in the tooth or teeth, or to perform one or more other tasks, such as imaging, profiling, or polishing, related to preparation of a dental restoration.

Though depicted as extending longitudinally on a top surface of block guide 104 in FIG. 3, the size, placement, depth, angle, and other characteristics of channel 120 on and in block guide 104, as well as for chamber 118, can be designed or customized according to a particular preparation, restoration, or patient. For example, if a restoration were needed on the side of a tooth, channel 120 could be formed on a side of block guide 104 proximate the area to be restored. In the example of FIG. 3, channel 120 may be arranged as depicted to restore a portion of the crown or top of a molar, while also providing visibility to the space between the molar and the adjacent tooth.

The designs of chamber 118 and channel 120 also can include its depth, length, width, and other characteristics of their configurations. For example, in FIG. 3 channel 120 is curved, which can accommodate a curved adaptor 108 and/or enable a dentist to manipulate handheld device 102 to slide and rotate within channel 120, thereby providing access to various angles and surfaces of the tooth or restoration area. In other embodiments, channel 120 can be round, non-curved, or have some other customization for one or more of a patient's tooth or teeth, a restoration location, a restoration type, a type of dental handpiece 102, a type of tool 110, a configuration of adaptor 108, or some other factor or characteristic.

Tool aperture 122 formed within channel 120 also can be customized in conjunction with channel 120 and adaptor 108. While channel 120 is formed to accommodate adaptor 108, tool aperture 122 is formed to accommodate tool 110 such that tool 110 can be brought into proximity with the tooth or teeth of the patient in chamber 118 when dental handpiece 102 is manipulated to insert adaptor 108 into channel 120 of block guide 104, thereby inserting tool into tool aperture 122.

The data necessary to create block guide 104 (and adaptor 108, and/or other components in some embodiments) can be obtained from a dental impression, a digital scan, or some other form or image of the relevant tooth or teeth, or data related to the tooth or teeth. In embodiments, block guide 104 can be derived from an optimized design of the preparation/restoration. For example, channel 120 can be reverse engineered by digitally placing tool 110 with adaptor 108 on the preparation surface and tracing the paths and surface covered by adaptor 108 while tool 110 scans the preparation surface. The design and shape optimization of the preparation, restoration, and block guide 104 are conducted using CAD/CAM and finite element analysis (FEA) software in conjunction with an internal or external shape optimization routine to minimize interfacial and bulk stresses.

FEA can be a cost-effective way to analyze the overall stress distributions in different dental structures and to predict their potential failures. Different designs and structures can be modeled, evaluated, and compared before fabrication and testing using FEA, but conventional approaches that do not also incorporate shape-optimization can result in repeated design modifications and numerical analyses through a trial-and-error process, which is time-consuming and inefficient. Thus, embodiments disclosed herein that additionally incorporate internal or external shape optimization routines can predict and thereby minimize interfacial and bulk stresses, addressing the aforementioned inefficiencies and provide an improved clinical result. In various embodiments, predicted stress distributions can be used as feedback to iteratively but automatically modify restoration designs.

After the designs of the preparation and restoration are shape optimized, such as by using FEA software, preparation guide system 100 can be designed using CAD/CAM software. Block guide 104 (and adaptor 108, and/or other components in some embodiments) can be physically formed via three-dimensional (3D) printing, molding, or some other suitable process. Suitable materials for block guide 104 comprise polymers, ceramics, composites, metals, combinations of these materials, or other suitable materials, including materials that are biocompatible. In some embodiments, multiple preparation guides can be used in sequence to make preparations that are more complicated (e.g., with multiple curvatures at different locations).

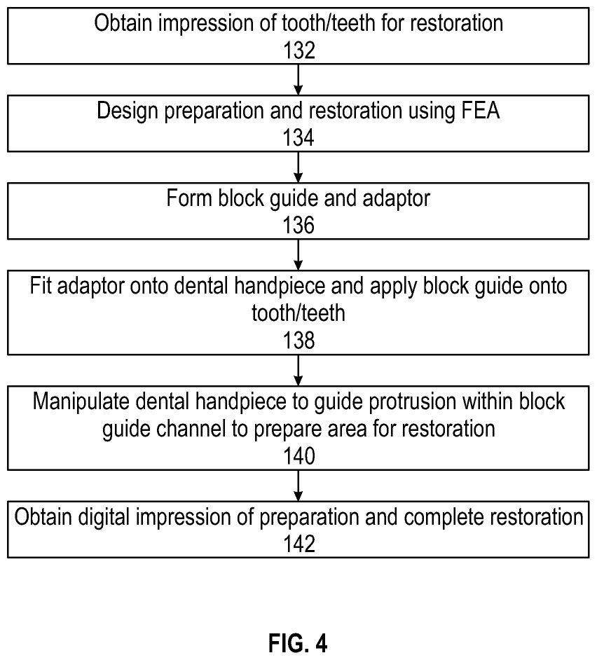

In use, and referring to FIG. 4, an impression of a tooth or teeth for restoration are obtained at 132. A shape-optimized preparation (i.e., by drilling or removing a portion of the tooth or teeth) and restoration are then designed using finite element analysis or another suitable technique at 134. With this data, a corresponding and customized block guide 104 (and adaptor 108 in some embodiments) can be formed for the shape-optimized restoration at 136. A dentist or other medical professional then fits adaptor 108 onto a dental handpiece 102 and applies block guide 104 onto a patient's tooth or teeth at 138. At 140, the dentist then manipulates dental handpiece 102 to move adaptor 108 within channel 120 of block device 104 such that tool 110 moves within tool aperture 122 and can be used to prepare the tooth or teeth for restoration. Once an impression (which can be digital or traditional) of the preparation is obtained at 142, the restoration itself can be adjusted, where necessary, and completed.

The number of impressions taken during the process will depend on the type of restoration to be made. For a direct restoration (e.g., filling a cavity), one impression typically will be made. For an indirect restoration (e.g., veneers, crowns, inlays, caps, or bridges), two impressions typically will be made. The first impression is of the unprepared tooth and the adjacent teeth so that block guide 104 can be made and the FEA can be performed. The second impression is of the cavity, taken after the cutting is completed, and is used to fabricate the indirect restoration itself. Although the cavity and the indirect restoration typically will be designed at the same time using FEA, it can be helpful to check the cut cavity (with a second impression) against what has been designed for the restoration.

The restoration itself can be prepared, such as by using industry-standard FEA or other algorithms and programs to customize and optimize the restoration for the patient, and the dentist or other practitioner can apply the restoration to the patient. In some embodiments, particularly direct restoration, block guide 104 and one or more dental handpieces 102 can be used to apply the restoration. In one particular example, a new block guide 104 can be created to assist in applying the restoration.

### EXAMPLE

The inventors conducted a study aimed to optimize the design of a posterior 2-unit cantilevered bridge which is attached to the abutment tooth via an inlay. The study considered both the abutment cavity design for the inlay as well as the fiber layout in the cantilever pontic.

Materials and Methods

Structural optimization of a 3-unit inlay-retained fiber-reinforced (FRC) fixed partial denture (FPD) was performed using a stress-induced material transformation (SMT) technique. Similar methods were used to optimize the 2-unit cantilevered FRC bridge in this study. Since the cantilevered design only has a single retainer, lowering the interfacial stresses at the abutment-retainer connection was first considered, prior to optimizing the FRC substructure in the prosthesis, to reduce the risk of debonding. Hence, a two-step approach was adopted for the structural optimization of the 2-unit inlay-retained cantilevered bridge.

Finite Element Model Construction

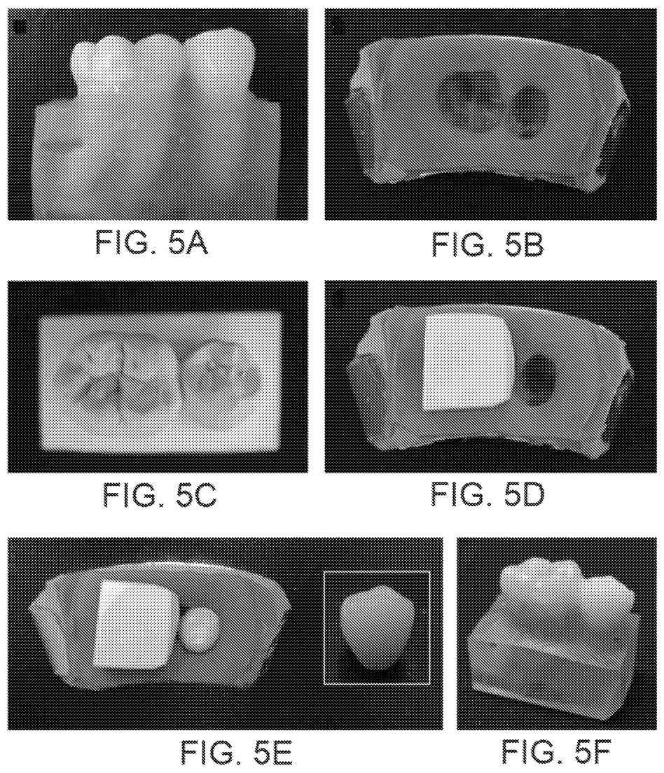

A human mandibular first molar and second premolar were embedded in orthodontic resin to form a physical model as shown in FIG. 5a. Then, a two-step (putty and wash) impression of this physical model was taken by using a partial tray (Kwik-Tray, Kerr, Brea, CA, USA) with vinyl polysiloxane impression material of first heavy-bodied consistency (e.g., Imprint™ 3 Quick Step Heavy Body) (injection type) and then light-bodied consistency (e.g., Imprint™ 3 Quick Step Regular Body) (injection type), as shown in FIG. 5b. An initial impression was made with only the heavy-bodied impression material to form a custom tray which provided a space for the secondary wash impression. After the heavy-bodied impression material had set, material within a perimeter approximately 2 mm away from the coronal part of the teeth was carved away to make space for the light-bodied impression material. The light-bodied material was syringed over this custom tray to make the secondary wash impression. A stone material (e.g., Die-Keen®) was then poured into the impression mold to form a working cast, as shown in FIG. 5c. The premolar portion was removed from the working cast with a cast trimmer and the remaining molar portion was put back into the impression mold, as shown in FIG. 5d. A resin composite (e.g., Filtek™ Z250 Universal Composite) was used to fill up the space for the premolar to fabricate a pontic without a retainer, as shown in FIG. 5e. Finally, the premolar pontic was attached to the mesial surface of the actual molar with resin cement (e.g., RelyX™ Ultimate Adhesive Resin Cement), as shown in FIG. 5f.

The whole assembly then was scanned by using a micro-CT scanner (e.g., with a XT H 255 by Nikon Metrology) with a tube voltage of 100 kV and a tube current of 170 μA. A total of 720 projections and four frames per projection were taken. The acquired images were reconstructed into a three-dimensional (3D) volume (e.g., using CT Pro 3D by Nikon Metrology). Subsequently, segmentation of the 3D volume was performed (e.g., using Avizo 6.0 by Visualization Sciences Group) to divide it into its constituent materials, i.e. enamel, dentin, bone, and resin composite. The 3D surfaces for each component were created and then exported as a stereolithography (STL) file for finite-element model construction (e.g., using Hypermesh 10.0 by HyperWorks-Altair Engineering).

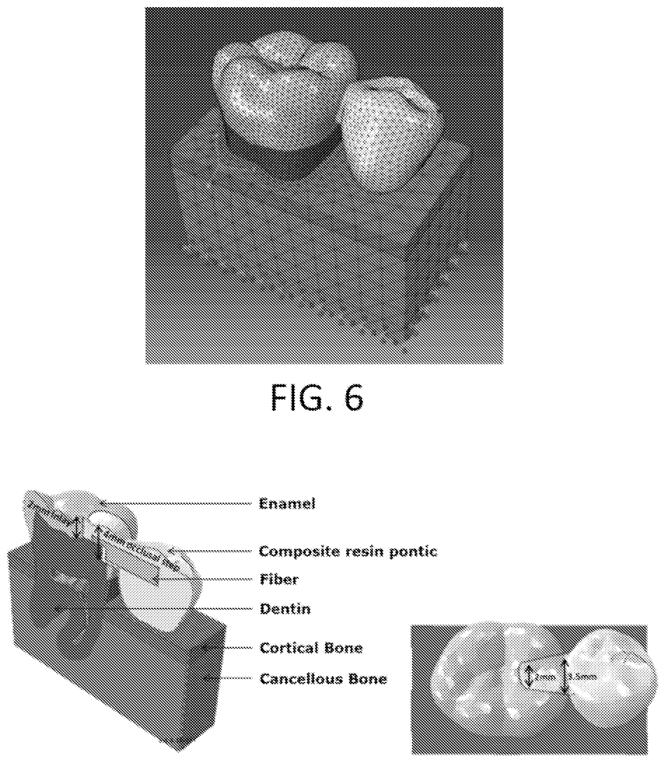

This study focused on the stress distributions within the restoration and the coronal portion of the abutment tooth. Therefore, the morphology of the mandibular jaw bone, which was sufficiently distant from the region of interest, did not need to be modeled accurately. Simply, and as shown in FIG. 6, a rectangular block of 11-mm thick with a top layer of 2-mm thick were used to simulate the surrounding cancellous and cortical bone, respectively. In addition, the mesh of the bone block was made coarser than those of the restoration and tooth structure to reduce computational cost. The entire finite element model contained 26,636 nodes and 134,863 4-node tetrahedral elements. All the interfacing components were assumed to be tied perfectly together. A concentrated force of 200 N was applied on the mesial fossa of the premolar pontic to simulate the worst chewing scenario. All displacements at the bottom of the bone block were fixed as boundary conditions.

Two-Step Structural Optimization

The structural optimization of the 2-unit cantilevered bridge was carried out in two steps (e.g., using ABAQUS 6.10-EF1 by Dassault Systemes Simulia) in conjunction with a user-defined material (UMAT) subroutine that defined the constitutive model of a material which was stress-state dependent. The first step was to obtain the optimal shape for the cavity preparation/retainer on the abutment by applying the subroutine to the tooth tissue only. In the second step, with a shape-optimized retainer, the subroutine was applied to the restoration to seek an optimal fiber layout for the FRC substructure.

In the first step, the enamel and dentin of the abutment tooth were assigned with a stress-dependent user-defined material. Thus, at every step increment, the UMAT subroutine was called to update the material properties of the tooth tissues using the predicted stresses from the previous increment. Initially, the material properties of these tooth tissues were given their natural values. Subsequently, their elastic moduli were modified iteratively based on the predicted local stresses in order to reduce the stresses at the base of the cantilever. Specifically, all the elements within the tooth with stresses larger than the assumed failure stress were given the properties of the “softer” resin composite. All the other parameters, including the material properties for the remaining tooth tissues and bones, the applied load, and the boundary conditions, were kept the same during the whole analysis. In this way, a retainer with reducing cohesive and interfacial stresses gradually grew from the pontic into the abutment tooth.

In the second step, an inlay retainer and the matching cavity preparation were created within the abutment tooth using the design derived in the first step. The remaining enamel and dentin were assigned with their natural material properties, while the restoration began as a uniform structure of resin composite and gradually acquired the user-defined material properties which now assumed those of the anisotropic unidirectional fibers. To obtain a suitable fiber-reinforcing layout within the restoration, regions with local stresses higher than the assumed failure stress were gradually replaced with the “stronger” fiber material. More importantly, the UMAT subroutine was able to align the fibers with the direction of the maximum principal stress. In this way, fiber reinforcement could be achieved most effectively.

There is a wide range of values reported for both the bond strength between teeth and restorations and the fracture strength of resin composites. A parametric study was therefore performed to understand the influence of the assumed bond strength on the shape of the cavity preparation, and that of the assumed fracture strength of the resin composite on the fiber layout. For both steps of the structural optimization, the assumed failure stress ranged from 5 to 30 MPa, in steps of 5 MPa. The stress-induced material transformation process continued iteratively until the results converged, i.e. when the interfacial or cohesive stress dropped below the assumed failure stress.

Comparison Between the Conventional and Optimized Designs

The designs provided by the optimization exercise may sometimes contain features that cannot be realized easily in practice. Therefore, when finalizing the optimized design, practical issues, such as a cavity shape that can actually be made, need to be considered, and simplifications or smoothing of the edges may have to be made. The final design for the optimized FRC bridge is thus a simplified and smoothed out version of that suggested by the structural optimization. As a comparison, a stepped box-shaped cavity/retainer was used for the conventional 2-unit cantilevered FRC dental bridge. The width of the box started at 2.5 mm in the mesial fossa of the first premolar and increased to 3.5 mm on the marginal ridge. The occlusal inlay was 2-mm thick and the occlusal step was 4-mm high. This is depicted in FIG. 7. The glass fibers were horizontally placed and fully surrounded with resin composite in the middle third of the connector and the mesial part of the pontic.

Stress analyses of the optimized and conventional designs were carried out to compare their mechanical performance numerically. To assess their resistance against retainer debonding and restoration fracture, the stresses at the tooth-restoration interface and those within the restoration were compared between the two designs.

Optimized Structure of the 2-Unit Cantilevered FRC Bridge

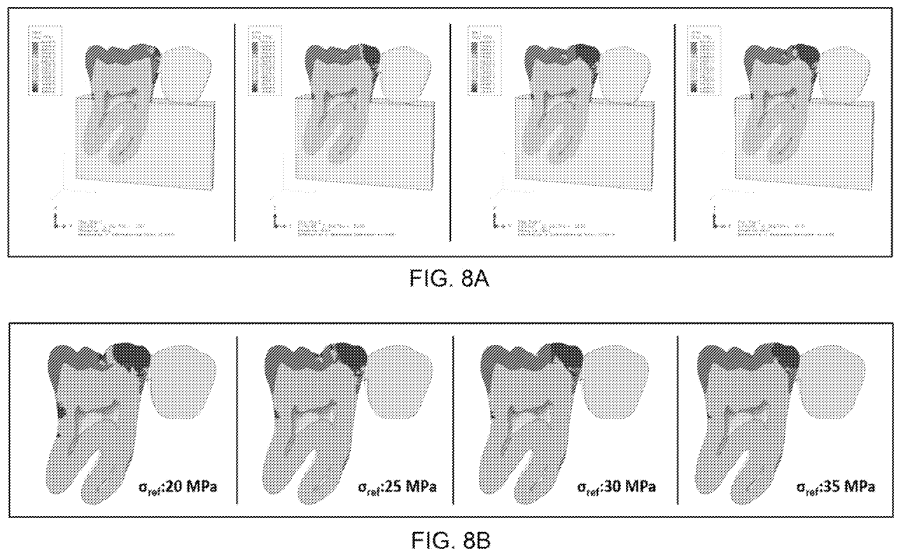

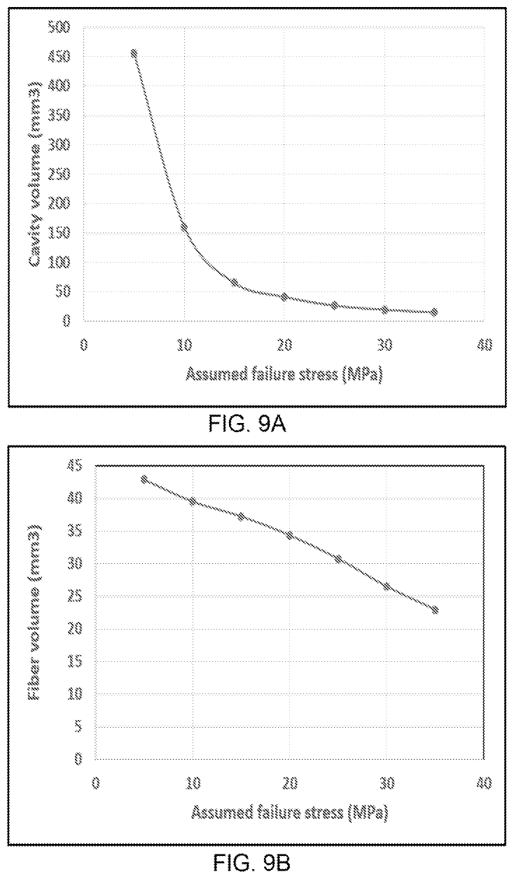

FIG. 8a shows the evolution and convergence of the cavity/retainer shape as given by the SMT optimization process for an assumed failure stress (σ_{ref}) of 25 MPa; and FIG. 8b shows the converged designs for different values of the failure stress. Irrespective of the assumed failure stress, the optimal design of the cavity/retainer has a shovel shape. However, the size of a cavity preparation increases exponentially with deceasing bond strength, as shown in FIG. 9a. In particular, the cavity preparation may need to be extended to the central fossa of the molar when the bond strength is lower than 20 MPa.

The results based on a failure stress of 25 MPa were used conservatively to construct the model for the second step of structural optimization. After smoothing the optimized retainer's contour (e.g., using Hypermesh 10.0 by HyperWorks-Altair Engineering), its size (26.1 mm³) was similar to that of the conventional box-shaped design (26.2 mm³). However, in terms of the area of bonding between the cavity and retainer, the optimized design is 17.5% smaller than the conventional one (31.3 mm² versus 38.0 mm²).

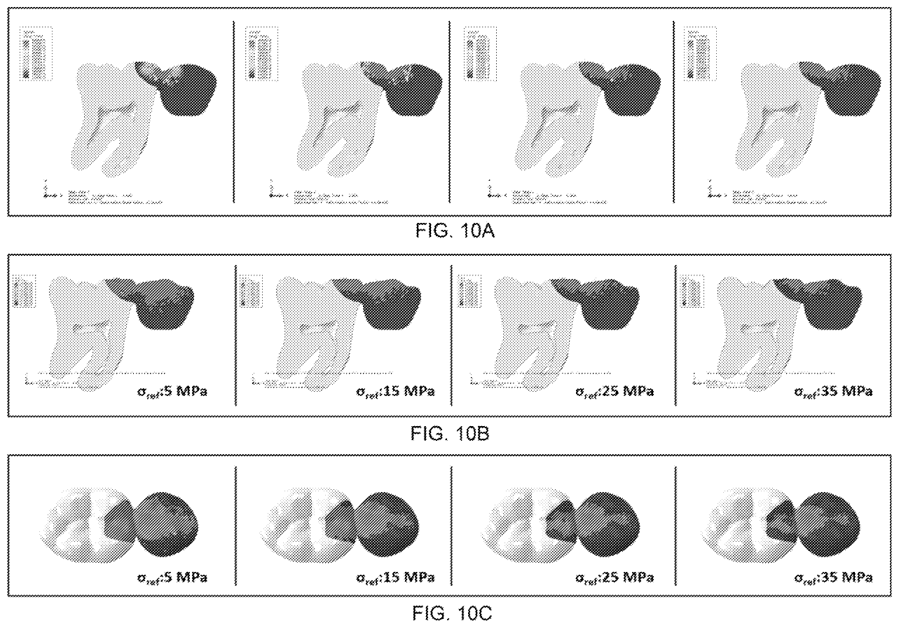

According to the results from the second step of the SMT optimization, fibers should be placed at the top of the connector region where tensile stress is highest, as shown in FIG. 10a. The volume of fibers required also increases with decreasing failure stress of the resin material, but the relationship is approximately linear, as shown in FIG. 9b. Irrespective of the assumed failure stress, the optimized fiber substructure takes up the shape of a bowtie, as can be seen in FIGS. 10b and 10c.

Comparison of Stresses Between Conventional and Optimized Designs

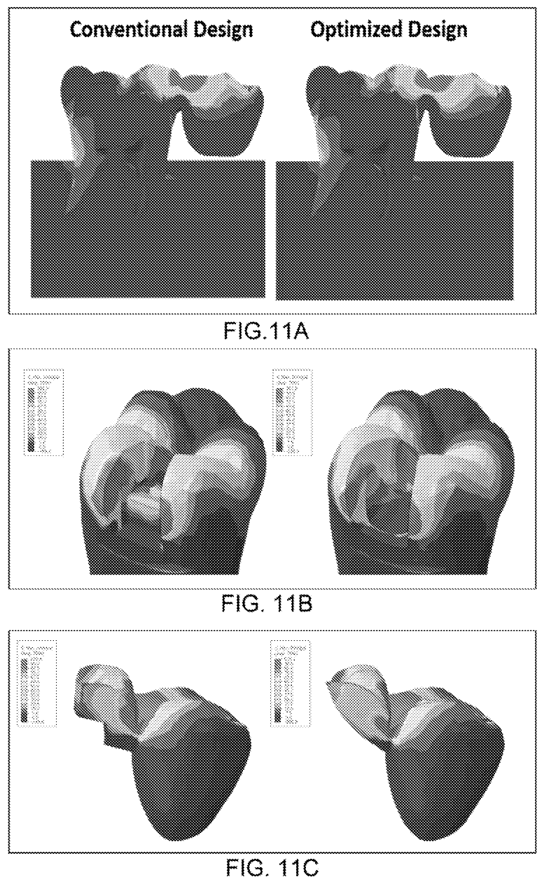

Finite element models without the fiber substructure were first used to evaluate the effect of the cavity shape on the stress distribution within the restored tooth. The optimized cavity of FIG. 10 was smoothed, as mentioned above. FIG. 11a shows the cross-sectional stress profiles of the two designs under a vertical load of 200 N at the mesial fossa of the second-premolar pontic. For both designs, the maximum principal stress concentrated at the buccoaxial and linguoaxial line angles, but the area of stress concentration was significantly reduced with the optimized cavity, which can be seen in FIG. 10b. The peak maximum principal stress within the tooth structure was also reduced from 381.7 MPa to 352.8 MPa. Without the fiber substructure, high tensile stresses can be seen in the occlusal third of the connector region for both conventional and optimized designs in FIG. 11c. However, the optimized cavity shape reduced the peak maximum principal stress within the restoration from 639.4 MPa to 525.4 MPa.

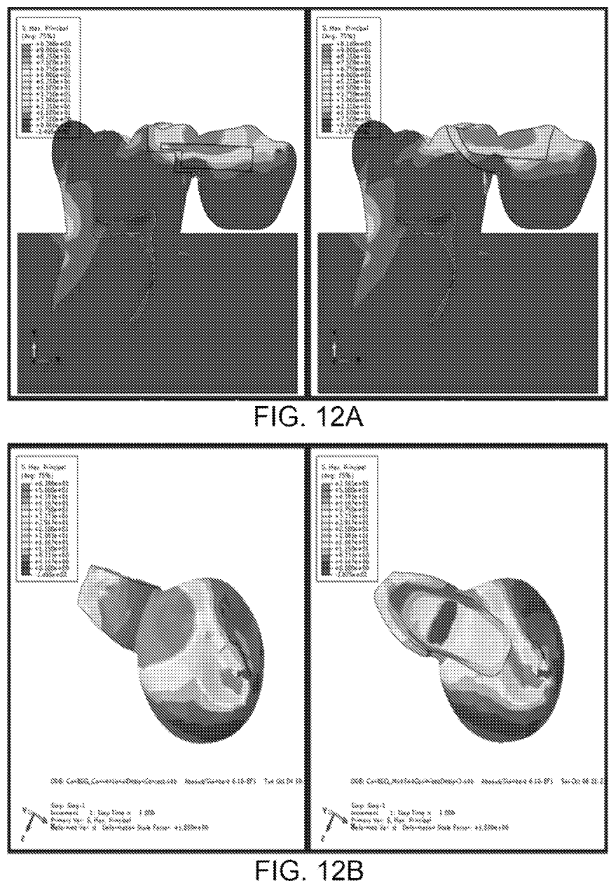

Referring to FIGS. 12a-b, in order to evaluate the influence of fiber position in the absence of other confounding factors, further comparison between the conventional and optimized designs were made with models containing similar embedded fiber volumes: 29.5 mm³ for the conventional versus 30.7 mm³ for the optimized (FIG. 12a). It would be difficult to fabricate bowtie-shaped fibers in practice. Therefore, a simple rectangular-box shape was adopted instead in the optimized design of the present study. In the optimized design, all the high tensile stresses are carried by the fiber substructure. In contrast, only the top half of the fiber substructure in the conventional design carries part of the high tensile stresses; the weaker veneering composite has to carry the remaining high tensile stresses. The optimized design reduces the peak maximum principal stress in the veneering composite, which locates in the connector, by about 45%, i.e., from 638.8 MPa to 356.5 MPa; see FIG. 9b. The peak interfacial tensile stress is located at the buccoaxial and linguoaxial wall in the conventional design and in the middle third of the shovel-shaped bottom in the optimized design, seen in FIG. 12a. Compared with the conventional box-shaped design, the shovel-shaped cavity preparation has roughly 70% reduction (189.6 MPa versus 57.04 MPa) in the maximum interfacial tensile stress. For the remaining tooth structure, the peak maximum principal stress concentrates around the cervical third of the proximal carvosurface margin and is reduced by about 30% in the optimized design (372.2 MPa versus 253.1 MPa; FIG. 12b).

The present study showed that the optimized design of the two-unit cantilevered FRC bridge has better mechanical performance than the conventional design for equal amounts of reinforcing fibers. Specifically, even with a smaller bonding area, the optimized cavity has much lower interfacial stresses (70% reduction), thus significantly reducing the risk of debonding of the pontic. Comparison between the stress profiles of the two designs further confirms that the optimized fiber substructure is better aligned with the maximum principal stress. Thus, by default, the normal and shear stresses at the resin-fiber interfaces are minimized to lower the risk of delamination. The much reduced maximum principal stress within the veneering resin of the optimized design (by 45%) is also expected to decrease the occurrence of cracking within the restoration.

It has been reported that inlay-retained FRC bridges often failed at lower loads than those retained by more extensive coverage of the abutment. However, the latter designs do not follow the spirit of minimally invasive dentistry. The optimized cavity design proposed here can preserve more tooth tissues by improving the retention for inlay-retained cantilevered bridges. The much shorter retainer margin is also expected to reduce the risk of secondary caries.

In this study, a bond strength of 25 MPa between the tooth and the restoration was selected so that the optimized design and the conventional one had a similar cavity size. This allowed the two cavity designs to be compared fairly. The actual bond strength between the tooth and resin composite can be higher than 25 MPa. Consequently, the size of the cavity preparation can be even smaller and more tooth tissue can be preserved. Clinically, the currently recommended dimensions of the inlay cavity are at least 2 mm wide by 2 mm deep. The cavity size derived from the SMT optimization based on a 25-MPa failure stress (σ_{Ref}) conforms to this clinical guideline. Despite the fact that the area of bonding is reduced by 17.5% due to the more rounded shovel shape, the lower interfacial stress of the optimized design is expected to improve the retention of the restoration.

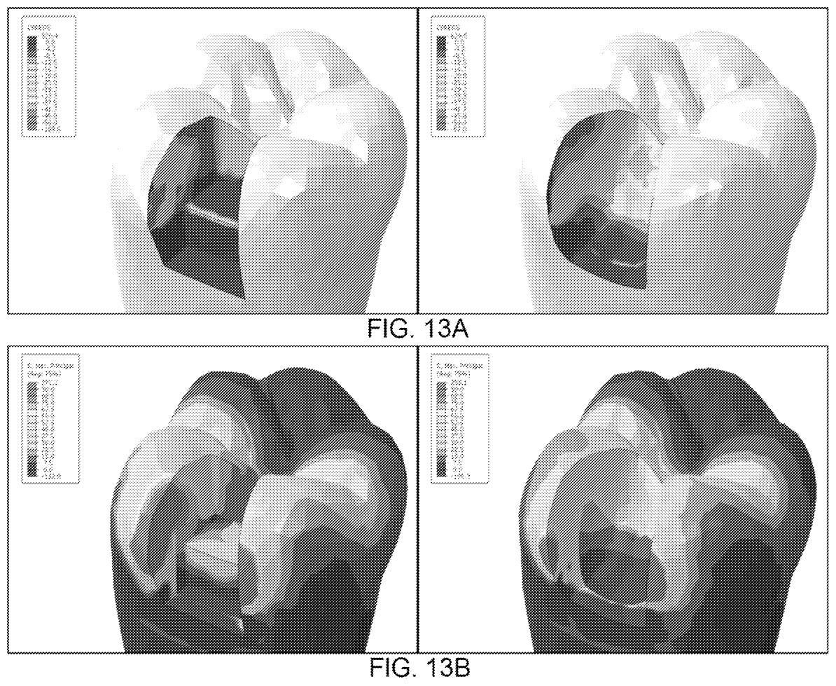

As shown in FIG. 13a, high interfacial tensile stresses concentrate on the labial and lingual axial walls of the cavity, particularly in the conventional design. Clinically, these are also the places where debonding usually initiates. Previous studies also reported tooth fracture as one of the failure modes in the inlay-retained restoration. This is because the intracoronal preparations weaken the tooth structure, especially those with the box-shaped design which has many stress-concentrating sharp line angles. In contrast, the optimized shovel-shaped cavity design has no sharp line angles. It can, therefore, reduce the maximum stress in the tooth by approximate 30% (FIG. 13b), and hence help lower the risk of tooth fracture.

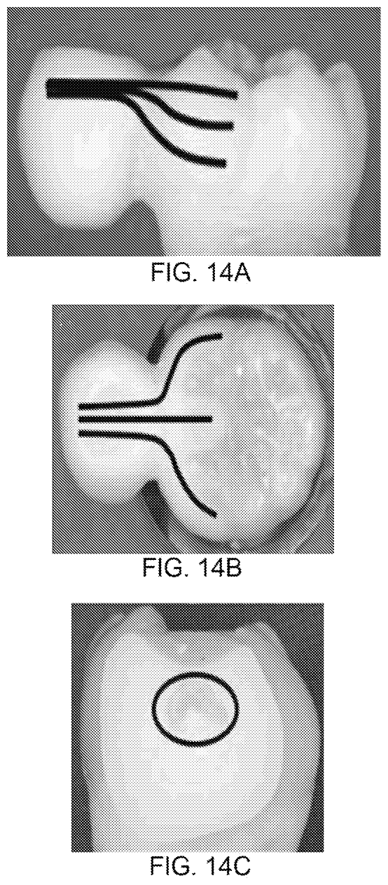

The SMT optimization suggested that the fibers be placed at the top of the connector. With this configuration, the high tensile stresses will be suitably borne by the fibers, thus reducing the maximum principal stress in the veneering composite by around 45%. FIG. 14 shows a similar fiber layout in an example conventional example. This design also places the fibers near the occlusal surface where high tensile stresses are expected. However, it also places fibers at lower positions in the inlay, which is not indicated by the optimization process. Thus, this example design may use more fibers than is required. The SMT optimization process also suggested that the fiber should have the shape of a bowtie (FIG. 10) to follow the maximum principal stress distribution more closely. However, it is difficult to fabricate bowtie-shaped fibers in practice. Therefore, a simple rectangular-box shape was adopted instead in the final design of the present study. Still, as mentioned, this simplified design can significantly reduce the maximum principal stress in the veneering composite when compared to the conventional design.

Clinically, the FRC cantilevered bridge restoration has been mainly used as an interim prosthesis, and most of the studies focused on its application in the anterior region. However, compared to 3-unit bridges, cantilevered bridges generally involve less tooth preparation and allow easier maintenance of oral hygiene. With these advantages and improved mechanical performance, the optimized FRC cantilevered bridge can be a viable long-term treatment option for both the anterior and posterior regions.

Thus, the present study by the inventors proposed a shovel-shaped retainer for the two-unit cantilevered FRC bridge where the reinforcing fibers are placed at the top of the connector area. With its lower interfacial and structural stresses, this optimized design is expected to outperform mechanically the conventional box-shaped design. This can potentially offer a more conservative treatment option for replacing the single missing tooth.

This study is an example, and the particular materials, tools, devices, and other factors mentioned therein are merely exemplary and not limiting with respect to the disclosure or claims. Those skilled in the art will recognize that suitable alternatives may be used, and that in some examples adjustments, adaptations, or substitutions may be needed in light of particular circumstances or factors.

Though embodiments discussed herein primarily related to dental restorations, other applications also are possible, such as dental procedures other than restorations, orthodontic procedures, other human medical treatments and therapies, veterinary procedures, and the like that involve the joining of different materials or components. Thus, even engineering application can be possible.

Embodiments disclosed herein can provide numerous advantages over conventional approaches to restorations and other dental and medical procedures. These include using shape optimization for both the preparation and restoration, as well as design and use of a block guide and adaptor, to provide restorations with more organic geometries that are subject to less interfacial and bulk stresses and therefore less likely to fail prematurely; and shortening visit length and the number of visits necessary to prepare and complete a restoration, to subject dentists, other practitioners, and patients to less generated aerosol, which is particular pertinent to the current COVID-19 pandemic but will also have applicability to reducing the transmission of other airborne illnesses.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A dental preparation block guide comprising:
a chamber that is customized to receive at least one tooth of a patient for a shape-optimized restoration of the at least one tooth; and
a channel formed in a surface of the block guide at a location selected relative to the shape-optimized restoration to be performed, the channel comprising an aperture extending through the surface to the chamber such that a dental handpiece can be inserted into and guided within the channel while a tool of the dental handpiece can interact with the at least one tooth in the chamber through the aperture to form a shape-optimized preparation in the at least one tooth to receive the shape-optimized restoration.

2. The dental preparation block guide of claim 1, further comprising an adaptor configured to be removably coupled to the dental handpiece to guide the dental handpiece within the channel.

3. The dental preparation block guide of claim 2, wherein the tool of the dental handpiece comprises a rotary cutting instrument.

4. The dental preparation block guide of claim 1, wherein the chamber is customized to receive the at least one tooth of the patient from a dental impression of the at least one tooth of the patient.

5. The dental preparation block guide of claim 4, wherein the dental impression is one of a physical impression or a digital impression.

6. The dental preparation block guide of claim 1, wherein the block guide is three-dimensionally printed, molded, or milled.

7. The dental preparation block guide of claim 1, wherein the shape-optimized preparation is shape-optimized using a finite element analysis.

8. The dental preparation block guide of claim 1, wherein the shape-optimized restoration is shape-optimized using a finite element analysis.

9. A method of providing a dental restoration in at least one tooth of a patient, the method comprising:
applying a finite element analysis operating on a computer system to a model of the at least one tooth of the patient to create a shape-optimized preparation in the at least one tooth of the patient; and
applying the finite element analysis operating on the computer system to the shape-optimized preparation in the at least one tooth of the patient to design a shape-optimized restoration to be applied to the shape-optimized preparation in the at least one tooth of the patient.

10. The method of claim 9, further comprising:
forming a dental preparation block guide comprising a customized chamber to fit onto the at least one tooth of the patient and a channel formed in a surface of the block guide at a location selected relative to the shape-optimized preparation to be performed, the channel comprising an aperture extending through the surface to the chamber;
inserting a dental handpiece into the channel such that a tool of the dental handpiece is inserted into the aperture of the channel;
guiding the dental handpiece within the channel such that the tool of the dental handpiece interacts with the at least one tooth in the chamber through the aperture to form the shape-optimized preparation in the at least one tooth of the patient.

11. The method of claim 10, further comprising applying the shape-optimized restoration to the shape-optimized preparation formed in the at least one tooth of the patient.

12. The method of claim 10, wherein forming the dental preparation block guide comprises:
obtaining a first impression of the at least one tooth of the patient;
forming the dental preparation block guide according to the first impression of the at least one tooth of the patient.

13. The method of claim 12, wherein obtaining the first impression of the at least one tooth of the patient comprises obtaining at least one of a physical impression or a digital impression.

14. The method of claim 12, further comprising creating a digital representation of the obtained first impression of the at least one tooth of the patient, wherein forming the dental preparation block guide further comprises three-dimensionally forming the dental preparation block guide according to the digital representation of the first impression of the at least one tooth of the patient.

15. The method of claim 12, further comprising:
obtaining a second impression of the tooth after forming the shape-optimized preparation in the at least one tooth of the patient; and
forming the shape-optimized restoration based on the second impression, wherein the shape-optimized restoration is an indirect restoration.

16. The method of claim 10, further comprising:
using a series of dental preparation block guides sequentially;
repeating the forming to prepare the series of dental preparation block guides; and
repeating the inserting and the guiding for each of the series of dental preparation block guides.

17. The method of claim 10, wherein forming the dental preparation block guide comprises three-dimensionally printing, molding, or milling the dental preparation block guide.

18. The method of claim 9, further comprising iteratively applying the finite element analysis on the computer system to refine at least one of the shape-optimized preparation or the shape-optimized restoration.
